SEQUENCE LISTING (1 sequence)

SEQ ID NO: 1 - protein, 303 aa, Artificial Sequence
  Description of Artificial Sequence Synthetic polypeptide
SKNFHLRPRDLISNINVIVLELKGSETTFMCEYADETATIVEFLNRWITFSQSIISTLTGGSSSTKKTQLQLEHLLLDLQMILNGINNYKNPKLTRMLTFKFYMPKKATELKHLQCLEEELKPLEEVLNLAQGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

(19) United States
(12) Patent Application Publication
(10) Pub. No.: US 2021/0371486 A1
(43) Pub. Date: Dec. 2, 2021

(54) IL-2 FUSION POLYPEPTIDE COMPOSITIONS AND METHODS OF MAKING AND USING THE SAME

(71) Applicant: Alkermes Pharma Ireland Limited, Dublin (IE)

(72) Inventors: Francisca O. Gbormittah, Acton, MA (US); Tarek A. Zeidan, Lexington, MA (US); Mayur Kalariya, Hopkinton, MA (US)

(21) Appl. No.: 17/315,973

(22) Filed: May 10, 2021

Related U.S. Application Data
(60) Provisional application No. 63/022,853, filed on May 11, 2020.

(51) Int. Cl. C07K 14/55; C07K 14/715; A61K 47/26; A61K 9/19

(52) U.S. Cl. CPC C07K 14/55; C07K 14/7155; A61K 38/00; A61K 9/19; A61K 47/26

(57) ABSTRACT

Provided herein are compositions comprising polypeptides comprising a circularly permuted interleukin-2 (IL-2) fused to the extracellular portion of an IL-2Rα chain, and methods of making and using such compositions.

### CROSS-REFERENCE TO RELATED APPLICATION

This application claims the benefit of U.S. Provisional Application Ser. No. 63/022,853, filed May 11, 2020, the entire disclosure of which is incorporated herein by reference.

### FIELD OF THE INVENTION

This disclosure relates to compositions comprising polypeptides comprising a circularly permuted interleukin-2 (IL-2) fused to the extracellular portion of an IL-2Rα chain, and methods of making and using such compositions.

### BACKGROUND

Polypeptides comprising a circularly permuted interleukin-2 (IL-2) fused to the extracellular portion of an IL-2Rα chain interleukin-2 (IL-2) interleukin-2 receptor alpha (IL-2Rα) hold great promise as anti-cancer agents. These polypeptides retain full ability to signal through the intermediate-affinity IL-2R complex that is expressed on memory CD8+ T cells and Natural Killer (NK) cells, but are sterically prevented from binding to the high-affinity IL-2R complex that is preferentially expressed on CD4+ FOXP3+ regulatory T cells (CD4+ Tregs) and endothelial cells. As a result of this selective IL-2R binding, the polypeptides selectively activate CD8+ T cells and NK cells, thereby promoting tumor cell killing. The inability to activate the high-affinity IL-2R on endothelial cells may also reduce the risk of toxicity due to capillary leak syndrome, a known risk of IL-2 therapies.

When used for the treatment of human subjects, the aforementioned polypeptides must be stored prior to use and transported to the point of administration. Reproducibly attaining a desired level of polypeptide in a subject requires that the polypeptide be stored in a formulation that maintains the bioactivity of the polypeptide. Accordingly, there is a need in the art for stable compositions of polypeptides. Preferably, such compositions will exhibit a long shelf-life, and be stable when stored and transported.

### SUMMARY

The present disclosure provides compositions comprising polypeptides comprising a circularly permuted IL-2 fused to the extracellular portion of an IL-2Rα chain, and methods of making and using such compositions. These compositions are specifically formulated to improve the stability and shelf-life of the polypeptides contained therein.

In one aspect, the disclosure provides a composition comprising:
a) about 1 mg to about 50 mg of a polypeptide comprising a circularly permuted IL-2 fused to the extracellular portion of an IL-2Rα chain;
b) sucrose;
c) citrate buffer; and
d) polysorbate 20.

In certain embodiments, the polypeptide comprises an amino acid having at least 95% identity to SEQ ID NO: 1. In certain embodiments, the polypeptide comprises the amino acid sequence of SEQ ID NO: 1. In certain embodiments, the polypeptide consists of the amino acid sequence of SEQ ID NO: 1.

In certain embodiments, the composition comprises about 100 mg to about 120 mg sucrose. In certain embodiments, the composition comprises about 110 mg sucrose.

In certain embodiments, the composition comprises about 4.0 mg to about 6.0 mg of citrate. In certain embodiments, the composition comprises about 5.0 mg citrate. In certain embodiments, the citrate buffer is formed by the combination of 2.03 mg/mL sodium citrate tribasic dihydrate and 0.97 mg/mL citric acid monohydrate in the aqueous solution. In certain embodiments, the citrate buffer is formed by the combination of 2.91 mg/mL sodium citrate tribasic dihydrate and 0.34 mg/mL citric acid monohydrate in the aqueous solution.

In certain embodiments, the composition comprises citric acid and sodium citrate tribasic dihydrate in a mass ratio of citric acid:sodium citrate tribasic dihydrate of between about 1:10 to about 1:2 (i.e., about 1:10, about 1:9, about 1:8, about 1:7, about 1:6, about 1:5, about 1:4, about 1:3, and about 1:2). In certain embodiments, the composition comprises citric acid and sodium citrate tribasic dihydrate in a mass ratio of citric acid:sodium citrate tribasic dihydrate of about 1:9. In certain embodiments, the composition comprises citric acid and sodium citrate tribasic dihydrate in a mass ratio of citric acid:sodium citrate tribasic dihydrate of about 1:2.

In certain embodiments, the composition comprises about 0.20 mg to about 0.24 mg polysorbate 20. In certain embodiments, the composition comprises about 0.22 mg polysorbate 20.

In certain embodiments, the composition comprises about 2.2 mg of the polypeptide. In certain embodiments, the composition comprises about 11 mg of the polypeptide. In certain embodiments, the composition comprises about 33 mg of the polypeptide.

In certain embodiments, the composition is a lyophilized cake. In certain embodiments, the dissolution of the lyophilized cake in water results in an aqueous solution with a pH of about 5.5 to about 6.5. In certain embodiments, the dissolution of the lyophilized cake in water results in an aqueous solution with a pH of about 6.1.

In certain embodiments, the dissolution of the lyophilized cake in water results in an aqueous solution with an osmolality of about 160 to about 230 mOsm/kg. In certain embodiments, the dissolution of the lyophilized cake in water results in an aqueous solution with an osmolality of about 179 mOsm/kg.

In certain embodiments, the dissolution of the lyophilized cake in a sodium chloride solution results in an aqueous solution with an osmolality of about 240 to about 340 mOsm/kg.

In certain embodiments, the aqueous solution is further diluted with water or a sodium chloride solution.

In certain embodiments, the aqueous solution comprises about 0.03 mg/mL of the polypeptide to about 0.2 mg/mL of the polypeptide.

In certain embodiments, the composition is an aqueous solution.

In certain embodiments, the composition is a 2.2 ml aqueous solution comprising 2.2 mg of the polypeptide. In certain embodiments, the composition is a 2.2 ml aqueous solution comprising 11 mg of the polypeptide. In certain embodiments, the composition is a 2.2 ml aqueous solution comprising 33 mg of the polypeptide. In certain embodiments, the composition is a 2.2 ml aqueous solution comprising 44 mg of the polypeptide.

In certain embodiments, the pH of the solution is about 6.1.

In certain embodiments, the composition is a single unit dose of the polypeptide.

In one aspect, the disclosure provides a composition comprising:
a) about 1 mg to about 50 mg (e.g., about 2, 11, or 33 mg) of a polypeptide comprising a circularly permuted IL-2 fused to the extracellular portion of an IL-2Rα chain;
b) about 100 mg to about 120 mg sucrose;
c) about 4.0 mg to about 6.0 mg of citrate anion; and
d) about 0.20 mg to about 0.24 mg polysorbate 20.

In certain embodiments, the polypeptide comprises an amino acid having at least 95% identity to SEQ ID NO: 1. In certain embodiments, the polypeptide comprises the amino acid sequence of SEQ ID NO: 1.

In certain embodiments, the composition comprises about 110 mg sucrose. In certain embodiments, the composition comprises about 5.0 mg citrate anion. In certain embodiments, the composition comprises about 0.22 mg polysorbate 20.

In certain embodiments, the composition comprises about 2.2 mg of the polypeptide. In certain embodiments, the composition comprises about 11 mg of the polypeptide. In certain embodiments, the composition comprises about 33 mg of the polypeptide. In certain embodiments, the composition comprises about 44 mg of the polypeptide.

In certain embodiments, the composition is a lyophilized cake. In certain embodiments, the lyophilized cake comprises:
a) about 2.2 mg of a polypeptide comprising the amino acid sequence of SEQ ID NO: 1;
b) about 110 mg sucrose;
c) about 5.0 mg of citrate anion; and
d) about 0.22 mg polysorbate 20.
   In certain embodiments, the lyophilized cake comprises:
a) about 11 mg of a polypeptide comprising the amino acid sequence of SEQ ID NO: 1;
b) about 110 mg sucrose;
c) about 5.0 mg of citrate anion; and
d) about 0.22 mg polysorbate 20.
   In certain embodiments, the lyophilized cake comprises:
a) about 33 mg of a polypeptide comprising the amino acid sequence of SEQ ID NO: 1;
b) about 110 mg sucrose;
c) about 5.0 mg of citrate anion; and
d) about 0.22 mg polysorbate 20.

In certain embodiments, the composition is an aqueous solution. In certain embodiments, the composition is a 2.2 ml aqueous solution comprising:
a) about 2.2 mg of a polypeptide comprising the amino acid sequence of SEQ ID NO: 1;
b) about 110 mg sucrose;
c) about 5.0 mg of citrate anion; and
d) about 0.22 mg polysorbate 20.
   In certain embodiments, the composition is a 2.2 ml aqueous solution comprising:
a) about 11 mg of a polypeptide comprising the amino acid sequence of SEQ ID NO: 1;
b) about 110 mg sucrose;
c) about 5.0 mg of citrate anion; and
d) about 0.22 mg polysorbate 20.
   In certain embodiments, the composition is a 2.2 ml aqueous solution comprising:
a) about 33 mg of a polypeptide comprising the amino acid sequence of SEQ ID NO: 1;
b) about 110 mg sucrose;
c) about 5.0 mg of citrate anion; and
d) about 0.22 mg polysorbate 20.
   In certain embodiments, the pH of the aqueous solution is about 6.1.

In certain embodiments, the composition is a single unit dose of the polypeptide.

In another aspect, the disclosure provides an aqueous composition comprising:
a) about 1 mg/mL to about 20 mg/mL of a polypeptide comprising a circularly permuted IL-2 fused to the extracellular portion of an IL-2Rα chain;
b) about 45 mg/mL to about 55 mg/mL sucrose;
c) about 10 mM to about 20 mM (e.g., about 10 mM to about 13 mM) citrate; and
d) about 0.09 mg/mL to about 1.1 mg/mL polysorbate 20, wherein the pH of the solution is about 5.5 to about 6.5.

In certain embodiments, the polypeptide comprises an amino acid sequence having at least 95% identity to SEQ ID NO: 1. In certain embodiments, the polypeptide comprises the amino acid sequence of SEQ ID NO: 1.

In certain embodiments, the aqueous composition comprises about 50 mg/mL sucrose. In certain embodiments, the aqueous composition comprises about 12 mM citrate.

In certain embodiments, the aqueous composition comprises about 0.1 mg/mL polysorbate 20. In certain embodiments, the aqueous composition has a pH of about 6.1.

In certain embodiments, the composition comprises about 1 mg/mL of the polypeptide. In certain embodiments, the composition comprises about 5 mg/mL of the polypeptide. In certain embodiments, the composition comprises about 15 mg/mL of the polypeptide. In certain embodiments, the composition comprises about 20 mg/mL of the polypeptide.

In another aspect the instant disclosure provides an aqueous composition comprising:
a) about 1, 5, or 15 mg/mL of a polypeptide comprising the amino acid sequence of SEQ ID NO: 1;
b) about 50 mg/mL to about 55 mg/mL sucrose;
c) about 12 mM citrate buffer; and
d) about 0.1 mg/mL polysorbate 20, wherein the pH of the composition is about 6.1.

In certain embodiments, the aqueous composition comprises:
a) about 1 mg/mL of a polypeptide comprising the amino acid sequence of SEQ ID NO: 1;
b) about 50 mg/mL sucrose;
c) about 12 mM (e.g., 11.95 mM) citrate; and
d) about 0.1 mg/mL polysorbate 20, wherein the pH of the solution is about 6.1.

In certain embodiments, the aqueous composition comprises:
a) about 5 mg/mL of a polypeptide comprising the amino acid sequence of SEQ ID NO: 1;
b) about 50 mg/mL sucrose;
c) about 12 mM (e.g., 11.95 mM) citrate; and
d) about 0.1 mg/mL polysorbate 20, wherein the pH of the solution is about 6.1.

In certain embodiments, the aqueous composition comprises:
a) about 15 mg/mL of a polypeptide comprising the amino acid sequence of SEQ ID NO: 1;
b) about 50 mg/mL sucrose;
c) about 12 mM (e.g., 11.95 mM) citrate; and
d) about 0.1 mg/mL polysorbate 20, wherein the pH of the solution is about 6.1.

In another aspect, the disclosure provides an article of manufacture comprising any of the foregoing compositions. In certain embodiments, the article is a glass vial.

In another aspect, the disclosure provides a lyophilized composition made by lyophilizing any of the foregoing aqueous solutions.

In another aspect, the disclosure provides a method of making a lyophilized composition, the method comprising lyophilizing any of the foregoing aqueous solutions.

In another aspect, the disclosure provides a method of making an aqueous composition, the method comprising dissolving any of the foregoing lyophilized compositions in an aqueous solvent.

In certain embodiments, the aqueous solvent is water for injection. In certain embodiments, the aqueous solvent is a sodium chloride solution. In certain embodiments, the sodium chloride solution comprises about 0.1% NaCl to about 0.5% NaCl. In certain embodiments, the sodium chloride solution comprises about 0.12% NaCl to about 0.41% NaCl. In certain embodiments, the sodium chloride solution comprises about 0.02 M NaCl to about 0.07 M NaCl. In certain embodiments, the aqueous composition comprises an osmolality of about 240 to about 340 mOsm/kg.

In certain embodiments, the pH of the aqueous composition is adjusted to about 6.1. In certain embodiments, the pH of the aqueous composition is adjusted to about 6.1 with a base. In certain embodiments, the base is sodium hydroxide.

In certain embodiments, the aqueous composition is further diluted with an aqueous solution comprising about 1% (w/w) of a surfactant. In certain embodiments, the surfactant is polysorbate 20. In certain embodiments, the aqueous solution further comprises about 0.1% (w/w) citric acid monohydrate, 0.2% (w/w) sodium citrate tribasic dihydrate, and 98.7% (w/w) water for injection.

In certain embodiments, the composition comprises a pharmaceutical composition.

In another aspect, the disclosure provides a method of activating natural killer cells (NK) cells in a subject, the method comprising administering to the subject an effective amount of any of the foregoing compositions.

In another aspect, the disclosure provides a method of treating cancer in a subject in need thereof, the method comprising administering to the subject an effective amount of any of the foregoing compositions. In certain embodiments, the cancer is renal cell carcinoma, melanoma, ovarian cancer, or lung cancer. In certain embodiments, the cancer comprises a refractory solid tumor.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts activity of Polypeptide A in several formulations as a reconstituted product or a lyophilized product. Activity was measured in a pSTAT5 ELISA assay.

FIG. 2 depicts size exclusion chromatography results of Polypeptide A in several formulations as a lyophilized product.

FIG. 3 depicts size exclusion chromatography results of Polypeptide A in several formulations as a reconstituted product.

FIG. 4 depicts differential scanning calorimetry studies of Polypeptide A as a function of buffer composition. The Tm of unfolding results are shown. All error bars are standard deviation from triplicate experiments.

FIG. 5 depicts OD350 with time studies of Polypeptide A at 72° C. as a function of buffer composition. Average OD350 after 8 hours at 72° C. is shown. All error bars are standard deviation from triplicate experiments.

FIG. 6 depicts micro-flow imaging studies of Polypeptide A as a function of buffer composition after 3 days of shaking at 300 RPM. Total subvisible particle concentration is shown, and error bars are standard deviation from triplicate experiments.

FIG. 7 depicts micro-flow imaging studies of Polypeptide A as a function of shaking stress and buffer composition. Total subvisible particle concentration is shown and error bars are standard deviation from triplicate experiments.

### DETAILED DESCRIPTION

Provided herein are compositions comprising polypeptides comprising a circularly permuted IL-2 fused to the extracellular portion of an IL-2Rα chain, and methods of making and using such compositions.

The formulations disclosed herein provide improved stability and shelf-life of the polypeptides contained therein. In particular, the polypeptide product retains biological activity, including after being lyophilized in the recited formulations and reconstituted in water for injection (WFI) or a similarly acceptable diluent. Importantly, the formulations described herein have been designed to allow the lyophilized product to be reconstituted in WFI, with is readily available to a patient or healthcare provider.

The formulations provided herein also yield a lyophilized cake that has a preferred appearance. Specifically, the cake is intact (not fragmented), has little to no shrinkage from the container (e.g., a glass vial), and have an even, concave surface.

### Selected Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the terms “comprising,” “including,” “having,” and grammatical variants thereof are to be taken as specifying the stated features, integers, steps or components but do not preclude the addition of one or more additional features, integers, steps, components or groups thereof. These terms encompass the terms “consisting of” and “consisting essentially of.”

As used herein, the terms “circular permutation” and “circularly permuted” refer to the process of taking a linear protein, or its cognate nucleic acid sequence, and fusing the native N- and C-termini (directly or through a linker, using protein or recombinant DNA methodologies) to form a circular molecule, and then cutting (opening) the circular molecule at a different location to form a new linear protein, or cognate nucleic acid molecule, with termini different from the termini in the original molecule. Circular permutation thus preserves the sequence, structure, and function of a protein, while generating new C- and N-termini at different locations that results in an improved orientation for fusing a desired polypeptide fusion partner as compared to the original molecule.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of up to ±5%, including ±5%, ±1%, and ±0.1% from the specified value, as such variations are appropriate to perform the disclosed methods.

As used herein, the terms “treat,” “treated,” “treating,” or “treatment” include the diminishment or alleviation of at least one symptom associated or caused by the state, disorder or disease being treated.

As used herein, the term “effective amount” in the context of the administration of a therapy to a subject refers to the amount of a therapy that achieves a desired prophylactic or therapeutic effect.

As used herein, the term “patient,” “individual” or “subject” refers to a human or a non-human mammal. Non-human mammals include, for example, livestock and pets, such as ovine, bovine, porcine, canine, feline and murine mammals. In certain embodiments, the subject is a human.

### IL-2 Fusion Polypeptides

In one aspect, the instant disclosure provides compositions of polypeptides comprising a circularly permuted interleukin-2 (IL-2) fused to the extracellular portion of an IL-2Rα chain. The polypeptides employed in the compositions disclosed herein exhibit preferential binding to the intermediate-affinity IL-2R complex comprising IL-2Rβ and the common gamma chain, IL-2Rγ) relative to the high-affinity IL-2R complex (comprising IL-2Rα, IL-2Rβ, and IL-2Rγ), and behave as selective agonists of the intermediate-affinity IL-2R complex. The design and generation of such polypeptides is described in U.S. Pat. No. 9,359,415, which is hereby incorporated by reference in its entirety.

An exemplary polypeptide useful for inclusion in the compositions disclosed herein is set forth below in SEQ ID. NO:1:

(SEQ ID NO: 1)
SKNFHLRPRDLISNINVIVLELKGSETTFMCEYADETATIVEFLNRWITF
SQSIISTLTGGSSSTKKTQLQLEHLLLDLQMILNGINNYKNPKLTRMLTF
KFYMPKKATELKHLQCLEEELKPLEEVLNLAQGSGGGSELCDDDPPEIPH
ATFKAMAYKEGTMLNCECKRGFRRIKSGSLYMLCTGNSSHSSWDNQCQCT
SSATRNTTKQVTPQPEEQKERKTTEMQSPMQPVDQASLPGHCREPPPWEN
EATERIYHFVVGQMVYYQCVQGYRALHRGPAESVCKMTHGKTRWTQPQLI
CTG

Accordingly, in certain embodiments, the amino acid sequence of the polypeptide comprises the amino acid sequence of SEQ ID. NO: 1. In certain embodiments, the amino acid sequence of the polypeptide consists of the amino acid sequence of SEQ ID. NO: 1.

The skilled worker will appreciate that amino acid sequence variants of SEQ ID. NO: 1 can also be employed in the compositions disclosed herein. For example, in certain embodiments, the amino acid sequence of the polypeptide comprises or consists of an amino acid sequence having at least 80% (e.g., 80, 81, 82, 83, 84, 85, 86, 87, 88, 89, 90, 91, 92, 93, 94, 95, 96, 97, 98, or 99%) identity to the amino acid sequence of SEQ ID. NO:1. In certain embodiments, the amino acid sequence of the polypeptide comprises or consists of an amino acid sequence having at least 95% identity to the amino acid sequence of SEQ ID. NO:1.

The skilled worker will also appreciate that amino acid sequence of the polypeptides employed in the compositions disclosed herein can be derivatized or modified, e.g., pegylated, amidated, etc.

In certain embodiments, the amount of the polypeptide in a formulation is about 1 mg to about 50 mg (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 33 mg, about 40 mg, about 44 mg, about 45 mg, or about 50 mg). In certain embodiments, the amount of the polypeptide is about 2.2 mg. In certain embodiments, the amount of the polypeptide is about 11 mg. In certain embodiments, the amount of the polypeptide is about 33 mg. In certain embodiments, the amount of the polypeptide is about 44 mg.

In certain embodiments, the concentration of the polypeptide in an aqueous formulation is about 1 mg/mL to about 50 mg/mL. In certain embodiments, the concentration of the polypeptide is about 1 mg/mL to about 20 mg/mL (e.g., about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL, or about 50 mg/mL). In certain embodiments, the concentration of the polypeptide is about 1 mg/mL. In certain embodiments, the concentration of the polypeptide is about 5 mg/mL. In certain embodiments, the concentration of the polypeptide is about 15 mg/mL. In certain embodiments, the concentration of the polypeptide is about 20 mg/mL.

In certain embodiments, the aqueous formulation is further diluted with water or a sodium chloride solution, thereby reducing the concentration of the polypeptide in the formulation. In certain embodiments, the aqueous solution comprises about 0.03 mg/mL of the polypeptide to about 0.2 mg/mL of the polypeptide (e.g., about 0.03 mg/mL, about 0.04 mg/mL, about 0.05 mg/mL, about 0.06 mg/mL, about 0.07 mg/mL, about 0.08 mg/mL, about 0.09 mg/mL, about 0.10 mg/mL, about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, about 0.19 mg/mL, or about 0.2 mg/mL).

In certain embodiments, the aqueous formulation is further diluted with an aqueous solution comprising about 1% (w/w) of a surfactant. In certain embodiments, the surfactant is polysorbate 20. In certain embodiments, the aqueous solution further comprises about 0.1% (w/w) citric acid monohydrate, 0.2% (w/w) sodium citrate tribasic dihydrate, and 98.7% (w/w) water for injection.

### Excipients & Buffers

In certain embodiments, the compositions disclosed herein comprise one or more excipients and/or buffers.

As used herein, the term “excipient” refers to any non-therapeutic agent added to the composition or formulation to provide a desired consistency, viscosity, or stabilizing effect. Suitable excipients for use in the compositions disclosed herein can act, e.g., as viscosity enhancing agents, stabilizers, solubilizing agents, etc. The excipient can be ionic or non-ionic. Suitable ionic excipients include salts such as NaCl or amino acid components such as arginine-HCl. Suitable non-ionic excipients include sugars, for example, monosaccharides (e.g., fructose, maltose, galactose, glucose, D-mannose, sorbose, etc.); disaccharides (e.g., lactose, sucrose, trehalose, cellobiose, etc.); polysaccharides (e.g., raffinose, melezitose, maltodextrins, dextrans, starches, etc.); and sugar alcohols (e.g., mannitol, xylitol, maltitol, lactitol, xylitol sorbitol (glucitol), etc.). For example, the sugar may be sucrose, trehalose, raffinose, maltose, sorbitol or mannitol. Additionally or alternatively, the sugar may be a sugar alcohol or an amino sugar. In certain embodiments, the sugar is sucrose.

In certain embodiments, the amount of the excipient (e.g., sucrose) in a formulation is about 1 mg to about 150 mg (e.g., about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg). In certain embodiments, the amount of the excipient (e.g., sucrose) in a formulation is about 90 mg to about 130 mg. In certain embodiments, the amount of the excipient (e.g., sucrose) in a formulation is about 99 mg to about 121 mg. In certain embodiments, the amount of the excipient (e.g., sucrose) in a formulation is about 110 mg.

In certain embodiments, the concentration of the excipient (e.g., sucrose) in an aqueous formulation is about 1 mg/mL to about 100 mg/mL (e.g., about 1 mg/mL, about 10 mg/mL, about 20 mg/mL, about 30 mg/mL, about 40 mg/mL, about 45 mg/mL, about 50 mg/mL, about 55 mg/mL, about 60 mg/mL, about 70 mg/mL, about 80 mg/mL, about 90 mg/mL, or about 100 mg/mL). In certain embodiments, the concentration of excipient (e.g., sucrose) is about 30 mg/mL to about 70 mg/mL. In certain embodiments, the concentration of excipient (e.g., sucrose) is about 45 mg/mL to about 55 mg/mL. In certain embodiments, the concentration of the excipient (e.g., sucrose) is about 50 mg/mL.

Suitable buffering agents for use in the compositions disclosed herein include organic acid and salts, such as salts of citric acid, ascorbic acid, gluconic acid, carbonic acid, tartaric acid, succinic acid, acetic acid or phthalic acid; Tris, tromethamine hydrochloride, or phosphate buffer. In addition, amino acid components can also be used as buffering agents. Such amino acid component includes glycine, histidine, and methionine. In certain embodiments, the buffer is a citrate buffer. As used herein, the term “citrate buffer” refers to a pH buffering system (in aqueous or lyophilized form) that utilizes citrate ions. Citrate buffer can be made using any art recognized methods, including, by combining: (i) citric acid, trisodium citrate dihydrate, and citric acid monohydrate; or (ii) citric acid monohydrate, sodium phosphate dibasic, and citric acid. In certain embodiments, citrate buffer is made using sodium citrate dihydrate and citric acid.

In certain embodiments, the amount of the buffering agent (e.g., citrate) in the formulation is about 1 mg to about 10 mg (e.g., about 1 mg, about 2 mg, about 3 mg, about 4 mg, about 5 mg, about 6 mg, about 7 mg, about 8 mg, about 9 mg, about 10 mg). In certain embodiments, the amount of the buffering agent (e.g., citrate) is about 5.9 mg to about 7.2 mg (e.g., about 5.9 mg, about 6.0 mg, about 6.1 mg, about 6.2 mg, about 6.3 mg, about 6.4 mg, about 6.5 mg, about 6.6 mg about, 6.7 mg about 6.8 mg, about 6.9 mg, about 7.0 mg, about 7.1 mg, or about 7.2 mg). In certain embodiments, the amount of the buffering agent (e.g., citrate) is about 6.6 mg. In certain embodiments, the amount of the citrate anion in the buffering agent (e.g., citrate) is about 4.0 mg to about 6.0 mg. In certain embodiments, the amount of the citrate anion in the buffering agent (e.g., citrate) is about 5.0 mg.

In certain embodiments, the concentration of the buffering agent (e.g., citrate) in an aqueous formulation disclosed herein is about 1 mM to about 50 mM (e.g., about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, or about 50 mM). In certain embodiments, the concentration of the buffering agent (e.g., citrate) is about 11 mM to about 13 mM (e.g., about 11.1 mM, 11.2 mM, 11.3 mM, 11.4 mM, 11.5 mM, 11.6 mM, 11.7 mM, 11.8 mM, 11.9 mM, 12.1 mM, 12.2 mM, 12.3 mM, 12.4 mM, 12.5 mM, 12.6 mM, 12.7 mM, 12.8 mM, or 12.9 mM). In certain embodiments, the concentration of the buffering agent (e.g., citrate) is about 12 mM. In certain embodiments, the concentration of the buffering agent (e.g., citrate) is about 11.95 mM. In certain embodiments, the citrate buffer contains 2.03 mg/mL (6.90 mM) sodium citrate tribasic dihydrate and 0.97 mg/mL (5.05 mM) citric acid.

In certain embodiments, the compositions disclosed herein have a pH of about 5.0 to about 8.0, of about 5.5 to about 7.5, of about 5.0 to about 7.0, of about 6.0 to about 8.0, or of about 6.0 to about 7.0. In certain embodiments, the compositions have a pH of about 5.5 to about 6.5. In certain embodiments, the compositions have a pH of about 5.8 to about 6.4. In certain embodiments, the compositions have a pH of about 6.1. In certain embodiments, the pH of the composition is adjusted to a pH of about 6.1. In certain embodiment, the pH is adjusted with a base. In certain embodiments, the base is a hydroxide salt, such as sodium hydroxide (NaOH) or potassium hydroxide (KOH). In certain embodiments, the composition is an aqueous composition and the pH of the aqueous composition is adjusted to a pH of about 6.1

As used herein, the term “surfactant” refers to organic substances having amphipathic structures; i.e., they are composed of groups of opposing solubility tendencies, typically an oil-soluble hydrocarbon chain and a water-soluble ionic group. Surfactants can be classified, depending on the charge of the surface-active moiety, into anionic, cationic and dispersing agents for various pharmaceutical compositions and preparations of biological materials. Suitable surfactants for use in the compositions disclosed herein include non-ionic surfactants, ionic surfactants and zwitterionic surfactants. Typical surfactants for use with the invention include sorbitan fatty acid esters (e.g., sorbitan monocaprylate, sorbitan monolaurate, sorbitan monopalmitate), sorbitan trioleate, glycerine fatty acid esters (e.g., glycerine monocaprylate, glycerine monomyristate, glycerine monostearate), polyglycerine fatty acid esters (e.g., decaglyceryl monostearate, decaglyceryl distearate, decaglyceryl monolinoleate), polyoxyethylene sorbitan fatty acid esters (e.g., polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g., polyoxyethylene sorbitol tetrastearate, polyoxyethylene sorbitol tetraoleate), polyoxyethylene glycerine fatty acid esters (e.g., polyoxyethylene glyceryl monostearate), polyethylene glycol fatty acid esters (e.g., polyethylene glycol distearate), polyoxyethylene alkyl ethers (e.g., polyoxyethylene lauryl ether), polyoxyethylene polyoxypropylene alkyl ethers (e.g., polyoxyethylene polyoxypropylene glycol, polyoxyethylene polyoxypropylene propyl ether, polyoxyethylene polyoxypropylene cetyl ether), polyoxyethylene alkylphenyl ethers (e.g., polyoxyethylene nonylphenyl ether), polyoxyethylene hydrogenated castor oils {e.g., polyoxyethylene castor oil, polyoxyethylene hydrogenated castor oil), polyoxyethylene beeswax derivatives (e.g., polyoxyethylene sorbitol beeswax), polyoxyethylene lanolin derivatives (e.g., polyoxyethylene lanolin), and polyoxyethylene fatty acid amides (e.g., polyoxyethylene stearic acid amide); C 10-C 18 alkyl sulfates (e.g., sodium cetyl sulfate, sodium lauryl sulfate, sodium oleyl sulfate), polyoxyethylene C 10-C 18 alkyl ether sulfate with an average of 2 to 4 moles of ethylene oxide units added (e.g., sodium polyoxyethylene lauryl sulfate), and C1-C 18 alkyl sulfosuccinate ester salts (e.g., sodium lauryl sulfosuccinate ester); and natural surfactants such as lecithin, glycerophospholipid, sphingophospholipids (e.g., sphingomyelin), and sucrose esters of C 12-C 18 fatty acids. A composition may include one or more of these surfactants. In certain embodiments, the compositions disclosed herein comprise polyoxyethylene sorbitan fatty acid esters e.g., polysorbate 20, 40, 60 or 80. In certain embodiments, the compositions disclosed herein comprise polysorbate 20.

In certain embodiments, the concentration of the surfactant (e.g., polysorbate 20) in the formulation is about 0.1 mg to about 1 mg (e.g., about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, or about 1 mg). In certain embodiments, the concentration of the surfactant (e.g., polysorbate 20) is about 0.15 mg to about 0.3 mg (e.g., about 0.16 mg, about 0.17 mg, about 0.18 mg, about 0.19 mg, about 0.21 mg, about 0.22 mg, about 0.23 mg, about 0.24 mg, about 0.26 mg, about 0.27 mg, about 0.28 mg, or about 0.29 mg). In certain embodiments, the concentration of the surfactant (e.g., polysorbate 20) is about 0.20 mg to about 0.24 mg. In certain embodiments, the concentration of the surfactant (e.g., polysorbate 20) in an aqueous formulation is about 0.22 mg.

In certain embodiments, the concentration of the surfactant (e.g., polysorbate 20) in an aqueous formulation is about 0.01 mg/mL to about 1 mg/mL (e.g., about 0.01 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL). In certain embodiments, the concentration of the surfactant (e.g., polysorbate 20) is about 0.05 mg/mL to about 0.15 mg/mL (e.g., about 0.05 mg/mL, about 0.06 mg/mL, about 0.07 mg/mL, or about 0.08 mg/mL about 0.09 mg/mL, about 0.1 mg/mL, about 0.11 mg/mL, about 0.12 mg/mL, about 0.13 mg/mL, about 0.14 mg/mL, or about 0.15 mg/mL,). In certain embodiments, the concentration of the surfactant (e.g., polysorbate 20) is about 0.09 mg/mL to about 0.11 mg/mL. In certain embodiments, the concentration of the surfactant (e.g., polysorbate 20) in an aqueous formulation is about 0.1 mg/mL.

It will be understood to those of skill in the art that the components of the compositions and compositions of the present invention may be described by units other than mg/mL. For example, the components of the compositions and compositions of the present invention may be described in units of molarity. The components of the compositions and compositions of the present invention may be further described in units of weight or mass percent.

### Lyophilization

In one aspect, the instant disclosure provided lyophilized compositions (e.g., lyophilized cake) of the polypeptides disclosed herein, and methods of making the same.

Lyophilization generally includes three main stages: freezing, primary drying and secondary drying. Freezing is necessary to convert water to ice or some amorphous formulation components to the crystalline form. Primary drying is the process step when ice is removed from the frozen product by direct sublimation at low pressure and temperature. Secondary drying is the process step when bounded water is removed from the product matrix utilizing the diffusion of residual water to the evaporation surface. Product temperature during secondary drying is normally higher than during primary drying. See, Tang X. et al. (2004) “Design of freeze-drying processes for pharmaceuticals: Practical advice,” Pharm. Res., 21:191-200; Nail S. L. et al. (2002) “Fundamentals of freeze-drying,” in Development and manufacture of protein pharmaceuticals. Nail S L editors. New York: Kluwer Academic/Plenum Publishers, pp 281-353; Wang et al. (2000) “Lyophilization and development of solid protein pharmaceuticals,” M J Pharm., 203:1-60; Williams N A et al. (1984) “The lyophilization of pharmaceuticals; A literature review.” J. Parenteral Sci. Technol, 38:48-59; and WO 2010/148337 A1.

Because of the variations in temperature and pressure through the lyophilization process, an appropriate choice of excipients or other components such as stabilizers, buffering agents, bulking agents, and surfactants are needed to prevent the polypeptides disclosed herein from degradation (e.g., protein aggregation, deamidation, and/or oxidation) during freeze-drying and storage. The lyophilized compositions disclosed herein contain a particular combination of constituents allow for stable long-term storage of the polypeptides disclosed herein that comprise a circularly permuted interleukin-2 (IL-2) fused to the extracellular portion of an IL-2Rα chain

In another aspect, the disclosure provides a lyophilized composition made by lyophilizing any one of the aqueous compositions disclosed herein that comprise a circularly permuted interleukin-2 (IL-2) fused to the extracellular portion of an IL-2Rα chain. In certain embodiments, the lyophilized composition is a lyophilized cake. In certain embodiments, the lyophilized composition is made by lyophilizing any one of the aqueous compositions disclosed herein following the lyophilization protocol recited in Table 2, Table 3, or Table 4.

In another aspect, the disclosure provides a method of making a lyophilized composition, the method comprising lyophilizing any one of the aqueous compositions disclosed herein that comprise a circularly permuted interleukin-2 (IL-2) fused to the extracellular portion of an IL-2Rα chain. In certain embodiments, the method of making a lyophilized composition comprises following the lyophilization protocol recited in Table 2, Table 3, or Table 4.

In another aspect, the disclosure provides a method of making an aqueous composition, the method comprising dissolving in an aqueous solvent any one of the lyophilized compositions disclosed herein that comprise a circularly permuted interleukin-2 (IL-2) fused to the extracellular portion of an IL-2Rα chain. In certain embodiments, the lyophilized composition is a lyophilized cake. In certain embodiments, the lyophilized composition is dissolved in 2.2 ml of water. In certain embodiments, the lyophilized composition is dissolved in a sodium chloride solution. In certain embodiments, the sodium chloride solution comprises about 0.1% NaCl to about 0.5% NaCl. In certain embodiments, the sodium chloride solution comprises about 0.12% NaCl to about 0.41% NaCl. In certain embodiments, the sodium chloride solution comprises about 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, or 0.50% NaCl. In certain embodiments, the sodium chloride solution comprises about 0.02 M NaCl to about 0.07 M NaCl. In certain embodiments, the sodium chloride solution comprises about 0.02 M, 0.03 M, 0.04 M, 0.05 M, 0.06 M, or 0.07 M NaCl. In certain embodiments, the aqueous composition comprises an osmolality of about 240 to about 340 mOsm/kg when dissolved in a sodium chloride solution. In certain embodiments, the aqueous composition dissolved in a sodium chloride solution may be administered to a patient via subcutaneous administration.

### Uses of Polypeptide Compositions

The compositions disclosed herein are particularly useful for the treatment, prevention, or amelioration of any disease or disorder associated with Interleukin 2 receptor signaling.

In one aspect, provided is a method of activating natural killer cells (NK) cells in a subject, the method comprising administering to the subject an effective amount of any one of the compositions disclosed herein that comprise a circularly permuted IL-2 fused to the extracellular portion of an IL-2Rα chain

In another aspect, provided is a method of treating cancer in a subject in need thereof, the method comprising administering to the subject an effective amount of any one of the compositions disclosed herein that comprise a circularly permuted IL-2 fused to the extracellular portion of an IL-2Rα chain. Cancers suitable for treatment using the composition disclosed herein include renal cell carcinoma, melanoma, ovarian cancer, and lung cancer. In certain embodiments, the cancer comprises a refractory solid tumor.

In certain embodiments, the composition is administered intravenously.

In certain embodiments, the composition is administered intravenously at a dose of about 1 μg/kg to about 15 μg/kg. In certain embodiments, the composition is administered intravenously at a dose of about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, or about 15 μg/kg.

In certain embodiments, the composition is administered intravenously each day for five consecutive days.

In certain embodiments, the composition is administered intravenously each day for five consecutive days, followed by nine consecutive days without intravenous administration of the composition.

In certain embodiments, the composition is administered intravenously each day for five consecutive days, followed by sixteen consecutive days without intravenous administration of the composition.

In certain embodiments, the composition is administered with 1) a first cycle comprising intravenous administration of the composition each day for five consecutive days, followed by nine consecutive days without intravenous administration of the composition; and 2) a second cycle comprising intravenous administration of the composition each day for five consecutive days, followed by sixteen consecutive days without intravenous administration of the composition.

In certain embodiments, the method comprises at least one additional cycle of administration, wherein each additional cycle comprises intravenous administration of the composition each day for five consecutive days, followed by sixteen consecutive days without intravenous administration of the composition.

In certain embodiments, the composition is administered subcutaneously.

In certain embodiments, the method comprises subcutaneous administration of the aqueous composition described above comprising an osmolality of about 240 to about 340 mOsm/kg.

In certain embodiments, the composition is administered subcutaneously at a dose of about 1 mg to about 15 mg. In certain embodiments, the composition is administered subcutaneously at a dose of about 1 mg. In certain embodiments, the composition is administered subcutaneously at a dose of about 2 mg. In certain embodiments, the composition is administered subcutaneously at a dose of about 3 mg. In certain embodiments, the composition is administered subcutaneously at a dose of about 4 mg. In certain embodiments, the composition is administered subcutaneously at a dose of about 5 mg. In certain embodiments, the composition is administered subcutaneously at a dose of about 6 mg. In certain embodiments, the composition is administered subcutaneously at a dose of about 7 mg. In certain embodiments, the composition is administered subcutaneously at a dose of about 8 mg. In certain embodiments, the composition is administered subcutaneously at a dose of about 9 mg. In certain embodiments, the composition is administered subcutaneously at a dose of about 10 mg. In certain embodiments, the composition is administered subcutaneously at a dose of about 11 mg. In certain embodiments, the composition is administered subcutaneously at a dose of about 12 mg. In certain embodiments, the composition is administered subcutaneously at a dose of about 13 mg. In certain embodiments, the composition is administered subcutaneously at a dose of about 14 mg. In certain embodiments, the composition is administered subcutaneously at a dose of about 15 mg.

In certain embodiments, the composition is administered subcutaneously once a week (Q1W), once every two weeks (Q2W), or once every three weeks (Q3W).

In certain embodiments, the composition is administered subcutaneously at a dose of about 1 mg to about 15 mg once a week (Q1W), once every two weeks (Q2W), or once every three weeks (Q3W).

In certain embodiments, the composition is administered subcutaneously at a dose of about 3 mg once a week (Q1W). In certain embodiments, the composition is administered subcutaneously at a dose of about 6 mg once every three weeks (Q3W).

In certain embodiments, the melanoma is one or both of mucosal melanoma or advanced cutaneous melanoma.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

The invention is further illustrated by the following examples, which should not be construed as further limiting. The practice of the present invention will employ, unless otherwise indicated, conventional techniques of organic synthesis, cell biology, cell culture, molecular biology, transgenic biology, microbiology and immunology, which are within the skill of the art.

### Example 1—Design and Testing of Polypeptide Compositions

In an effort to determine the optimal formulation for Polypeptide A (a circularly permuted IL-2 fused to the extracellular portion of an IL-2Rα chain comprising the amino acid of SEQ ID NO: 1), several formulations of Polypeptide A were tested for their effects on protein stability, pH stability, physio-chemical behavior, lyophilized cake uniformity, and resistance to adhering to the storage vial post-lyophilization. Table 1 below recites the specific components and their concentrations for each formulation tested. The different compositions were subjected to one of the lyophilization cycle protocols recited in tables 2-4 below.

TABLE 1
Constituents of Polypeptide A formulations

| Component | Formulation 1 | | Formulation 2 | | Formulation 3 | |
|---|---|---|---|---|---|---|
| Bulking Excipient | Sucrose | | Mannitol | | Sucrose | |
| (Conc.) | (50 mg/mL) | | (50 mg/mL) | | (50 mg/mL) | |
| Buffer (Conc.) | Glycine | | Glycine | | Histidine | |
| | (10 mg/mL) | | (10 mg/mL) | | (10 mg/mL) | |
| Surfactant | Polysorbate 20 | | Polysorbate 20 | | Polysorbate 20 | |
| (Conc.) | (0.1 mg/mL) | | (0.1 mg/mL) | | (0.1 mg/mL) | |
| Buffer pH | 5.9 | | 5.7 | | 6.5 | |
| Density | 1.020 | g/mL | 1.021 | g/mL | 1.018 | g/mL |
| pH post filtration | 6.6 | | 6.6 | | 6.5 | |
| Conc. of Polypeptide A | 0.75 | mg/mL | 0.96 | mg/mL | 0.79 | mg/mL |
| post filtration | | | | | | |
| Component | Formulation 4 | | Formulation 5 | | Formulation 6 | |
| Bulking Excipient | Sucrose | | None | | Sucrose | |
| (Conc.) | (50 mg/mL) | | | | (50 mg/mL) | |
| Buffer (Conc.) | Sodium Citrate | | Succinic Acid | | Methionine | |
| | (3 mg/mL) | | (40 mg/mL) | | (10 mg/mL) | |
| Surfactant | Polysorbate 20 | | Polysorbate 20 | | Polysorbate 20 | |
| (Conc.) | (0.1 mg/mL) | | (0.1 mg/mL) | | (0.1 mg/mL) | |
| Buffer pH | 6.3 | | 5.5 | | 5.9 | |
| Density | 1.015 | g/mL | 1.021 | g/mL | 1.011 | g/mL |
| pH post filtration | 6.1 | | 5.4 | | 6.3 | |
| Conc. of Polypeptide A | 1.03 | mg/mL | 1.00 | mg/mL | 0.85 | mg/mL |

post filtration

TABLE 2
Lyophilization cycle - 2 mg vial

| | | | | Chamber |
|---|---|---|---|---|
| | | Temperature | Time Set point | Pressure Set |
| | | Set point (° C.) | (hours) (±0.5 | point (μmHg) |
| Segment | Type | (±5° C.) | hours) | (±20 μmHg) |
| 1 | Hold | 5 | 4.0 | ≤14.7 psia |
| 2 | Ramp | −50 | 3.7 | ≤14.7 psia |
| 3 | Hold | −50 | 7.0 | ≤14.7 psia |
| 4 | Evacuation | −50 | | 40 |
| 5 | Ramp | −28 | 0.7 | 40 |
| 6 | Hold | −28 | 60 | 40 |
| 7 | Ramp | 25 | 3.5 | 40 |
| 8 | Hold | 25 | 9 | 40 |
| 9 | Stopper | 25 | | 14.7 psia ± |
| | | | | 0.7 psia |

TABLE 3
Lyophilization cycle - 10 mg vial

| | | | | Chamber |
|---|---|---|---|---|
| | | Temperature | Time Set point | Pressure Set |
| | | Set point (° C.) | (hours) (±0.5 | point (μmHg) |
| Segment | Type | (±5° C.) | hours) | (±20 μmHg) |
| 1 | Hold | 5 | 4.0 | ≤14.7 psia |
| 2 | Ramp | −50 | 1.8 | ≤14.7 psia |
| 3 | Hold | −50 | 4.0 | ≤14.7 psia |
| 4 | Evacuation | −50 | | 30 |
| 5 | Ramp | −28 | 0.7 | 30 |
| 6 | Hold | −28 | 66 | 30 |
| 7 | Ramp | 25 | 3.5 | 30 |
| 8 | Hold | 25 | 9 | 30 |
| 9 | Stopper | 25 | | 14.7 psia ± |
| | | | | 0.7 psia |

TABLE 4
Lyophilization cycle - 30 mg vial

| | | | | Chamber |
|---|---|---|---|---|
| | | Temperature | Time Set point | Pressure Set |
| | | Set point (° C.) | (hours) (±0.5 | point (μmHg) |
| Segment | Type | (±5° C.) | hours) | (±20 μmHg) |
| 1 | Hold | 5 | 4.0 | ≤14.7 psia |
| 2 | Ramp | −45 | 2.8 | ≤14.7 psia |
| 3 | Hold | −45 | 4.0 | ≤14.7 psia |
| 4 | Evacuation | −45 | | 50 |
| 5 | Ramp | −25 | 0.8 | 50 |
| 6 | Hold | −25 | 66 | 50 |
| 7 | Ramp | 30 | 3.1 | 50 |
| 8 | Hold | 30 | 8 | 50 |
| 9 | Stopper | 30 | | 14.7 psia ± |
| | | | | 0.7 psia |

### Physio-Chemical Tests

The physio-chemical behavior of Polypeptide A in formulations 1-6 was characterized at low temperatures by measuring electrical resistance, performing freeze drying microscopy, and performing differential scanning calorimetry.

### Electrical Resistance

Resistance was measured on approximately 1.8 mL of sample in a 20 mm glass sample tube using a 0 to 20 megaohm resistance instrument and a ceramic resistance probe consisting of two parallel gold strips on a ceramic plate. Temperature was measured using a 32-gauge type-T thermocouple located at the ceramic probe on the opposite side of the gold plates. The measurement and recording instruments were calibrated across the temperature range used for analysis using National Institute of Standards and Technology (NIST) traceable reference standards. Accuracy of the system was verified using a standard reference solution.

Using the standard method, the material was cooled and warmed at an average controlled rate of 0.5° C. per minute to assess its thermal characteristics in order to establish a material-specific phase transition. The resistance sample was analyzed at atmospheric conditions. A deviation in resistance was used to determine an onset of the phase transition upon warming. Temperature measurements were recorded every ten seconds during the entire analysis using a Kaye Validator instrument with the Collect program.

### Freeze Drying Microscopy

Approximately 0.15 mL of solution was dispensed into a glass cell. The cell was then placed on a temperature-controlled freeze-drying stage. The sample cell was outfitted with two 32-gauge type-T thermocouples placed directly into the material at the bottom and center of the cell to monitor sample temperatures. The liquid sample was cooled at an average controlled rate of 0.5° C. per minute to a target set point of −50° C. or below. Upon the completion of freezing, the stage chamber was evacuated to initiate sublimation of ice. The stage was then warmed at an average controlled rate of 0.5° C. per minute during sublimation and drying. Behavior of the sample during freezing and drying was observed using an Infinivar Microscope capable of magnification from 16 to 330× coupled to a Super WDR CCD Camera. Observed changes in the frozen and dried portions of the sample reflective of the material exceeding a phase transition were correlated with the sample temperature. Temperature measurements were recorded every ten seconds during the complete analysis using a Kaye Validator instrument with the Collect program.

Differential Scanning calorimetry

Low temperature differential scanning calorimetry (LT DSC) was used in conjunction with the analyses described above as a means of assessing physio-chemical behavior during freezing and warming for determining phase transitions. LT DSC analysis followed the current USP<891> and was performed using a TA Instruments Q200 DSC. The TA Instruments Q200 DSC was operated with a Refrigerated Cooling System. Test parameters were implemented using TA Advantage software (v5.0.4). The scan data was recorded and graphed using the TA Universal Analysis software (v4.5A). A sample size of 19.4 mg of solution was placed in an aluminum sample pan with a lid crimped in place. Nitrogen, NF, was used to purge the sample continuously at a flow rate of 50 ml/minute. The instrument was calibrated per Lyophilization Technology, Inc.'s calibration program. During cooling and warming, evolution or uptake of heat for the sample reflects the differences in energy as the sample undergoes a thermal event. This difference in heat energy is recorded for analysis of the results.

### Physio-Chemical Behavior Characterization Results

The results of the physio-chemical behavior characterization revealed that formulation 4 led to a lyophilized cake with a uniform appearance that remained intact upon inversion and jarring, minimal residual material adhered to the sides and bottom of the vial upon lyophilization, and had the highest glass transition property. The results for formulation 4 were superior to all other formulations tested. Furthermore, the other tested formulations failed to yield a stable lyophilization cycle for producing the lyophilized Polypeptide A composition.

### Stability Tests

The stability of Polypeptide A in formulations 1-6 was characterized using capillary isoelectric focusing (cIEF), size-exclusion high-performance liquid chromatography (HPLC), SDS-PAGE, and activity assays.

Capillary Isoelectric Focusing (cIEF)

cIEF was used to measure the presence of charge variants in the tested formulations. The drug product sample was prepared by reconstituting the sample in 2.2 mL water for injection (WFI). The sample was visually inspected to ensure that contents were free of visible particulates. Reference standards, samples, and blanks were prepared for analysis by preparing a master mix by calculating the volume of each reagent required from the table below using the following equation: “volume of reagent”=[(“n+2”)×“V”] Where: n=number of samples; V=volume (4) of reagent per sample (see Table 5 below).

TABLE 5
Reagent mixture for cIEF analysis

| Reagent (200 μL total volume) | V (μL/Sample) |
|---|---|
| 1% Methyl Cellulose (0.35% final concentration) | 70 |
| Pharmalytes 5-8 (3% final concentration) | 6 |
| Pharmalytes 3-10 (1% final concentration) | 2 |
| pI 5.12 Marker | 0.5 |
| pI 7.90 Marker | 0.5 |

The calculated volume of reagents was added to an appropriate size tube and vortexed to mix. The blank was prepared by transferring 158 μL of the master mix to a 1.5 mL microcentrifuge tube and 2424 of WFI was added to the tube and vortexed gently to mix. The Polypeptide A reference standard was prepared in the same manner as the blank. The sample was prepared by transferring 79 μL of the master mix to a 1.5 mL tube for each sample and 121 μL of sample was added to the tube and vortexed gently to mix. The injection sequence and the analytical conditions used are set forth in Tables 6 and 7, respectively. For analysis, all peaks between the pI markers are integrated. Results were calculated by reporting the pI and area % of all peaks ≥0.05%.

TABLE 6
Injection sequence used for cIEF analysis

| Sample ID | Number of Injections |
|---|---|
| Blank | 1 |
| Polypeptide A Reference | 1 |
| Standard | |
| Samples | 1 each |
| Polypeptide A Reference | 1 |
| Standard | |
| Blank | 1 |

TABLE 7
Analytical conditions used for cIEF analysis

| | |
|---|---|
| Tray Type: | 48 Vial Tray with 4 Rinse Vials |
| Tray Temp: | 5.0° C. |
| Focus Period 1: | 1500 V for 1.00 min |
| Focus Period 2: | 3000 V for 7.00 min |

### Size Exclusion HPLC

Size exclusion HPLC was used to measure purity in the tested formulations. The drug product sample was prepared by reconstituting the sample in 2.2 mL WFI. The sample was visually inspected to ensure that contents were free of visible particulates. Reference standards, samples, and blanks were tested for analysis using the following analytical conditions and injection sequence set forth in Tables 8 and 9, respectively.

TABLE 8
Analytical conditions used for HPLC analysis

| | |
|---|---|
| Mobile Phase | 50 mM Na Phosphate, 200 mM NaCl, pH 7.2 |
| Run Time | 15 minutes |
| Flow Rate | 1.0 mL/min |
| Column | TSKgel G2000SW_{XL}, 7.8 mm ID × 30 cm, 5 |
| | μm |
| Column Temp | 25° C. ± 5° C. |
| Sample Temp | 5° C. ± 3° C. |
| Detection | UV absorbance at 280 nm |
| Injection Volume | 100 μL |

TABLE 9
Injection sequence used for HPLC analysis

| | Number of |
|---|---|
| Sample ID | Injections |
| Mobile Phase | 1+ |
| Formulation Buffer Blank | 1 |
| Gel Filtration Standard (GFS) | 1 |
| Polypeptide A Reference Standard | 1 |
| Samples | 1 |
| Polypeptide A Reference Standard | 1 |
| Gel Filtration Standard (GFS) | 1 |

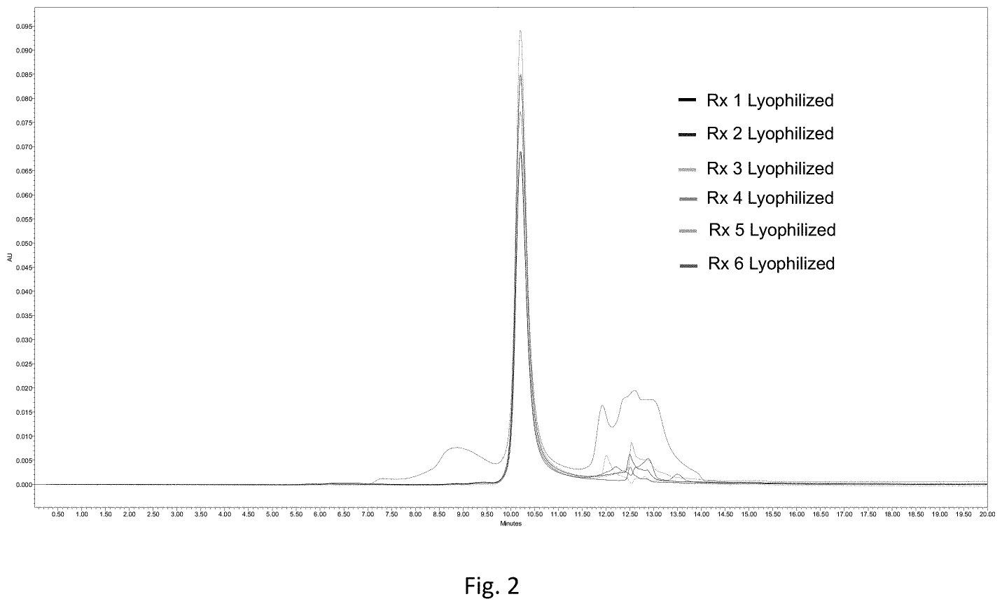

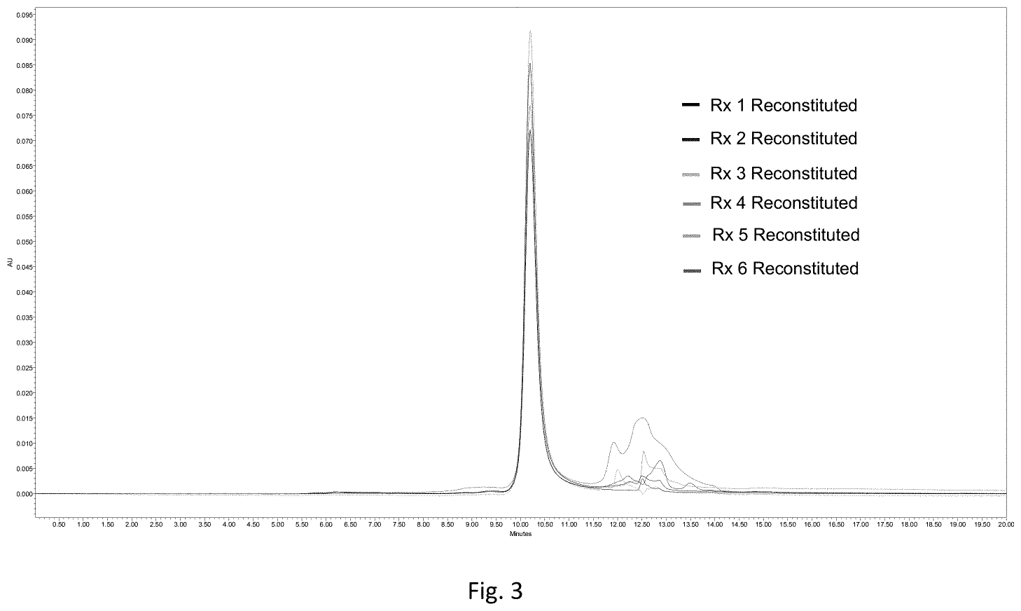

The results were reported as the area % and elution time of all integrated peaks greater than 0.05% area. FIG. 2 and FIG. 3 depict SEC peaks for each formulation tested.

### Cell-Based Activity Assay

The activity of Polypeptide A in each formulation was assessed by measuring binding of the formulations to HH cells (a human T lymphocyte cell line which have the βγIL2 receptor isoform present on their surface). Polypeptide A binding was measured by determining the amount of phosphorylated STATS (phospho-STATS or pSTATS) present in the HH cells after contact with each formulation, using an ELISA assay. The Invitrogen InstantOne ELISA phosphor-STATS alpha/beta (pTyr694/pTyr699) kit was used to perform the ELISA assay.

The drug product sample was prepared by reconstituting the sample in 2.2 mL WFI. The sample was visually inspected to ensure that contents were free of visible particulates.

A sample diluent was prepared by adding 25 mL of Fetal Bovine Serum (FBS) to 500 mL of Hanks Balanced Salt Solution (HBSS) for a final concentration of 5% FBS and warmed to 37° C. A wash buffer comprising Phosphate Buffered Saline (PBS) with 0.05% Tween 20 was used.

Samples and standards were diluted to final protein concentrations in the assay of 750 ng/mL, 250 ng/mL, 83 ng/mL, 28 ng/mL, 9.3 ng/mL, 3.1 ng/mL, 1.0 ng/mL, and 0.3 ng/mL. A stock solution of HH cells at a density of approximately 1.2×10⁶ cells/mL was prepared and 50 μl of the cell stock solution was added to each well of a 96-well plate that contained the diluted sample or standard. The cells were incubated at 37° C. for 30 minutes. After incubation, the cells were lysed in a cell lysis buffer for 10 minutes. After lysis, 50 μl of the lysed cell mix was transferred to an ELISA plate followed by 50 μl of phospho-STATS A/B antibody cocktail. The mix was then incubated for 1 hour then washed 3 times with wash buffer. 100 μl of a detection reagent was then added to each well and the plate was incubated for 15 minutes. 100 μl of a stop solution was then added to each well and the plate was read at 450 nm on a microplate reader.

Individual EC50 values were measured and % relative standard deviation (RSD) was calculated for the reference standard EC50 values and for the control EC50 values.

The geometric mean of the three EC50 values for reference standard (Ref Std EC50GM) and the geometric mean of three EC50 values for the control (Control EC50) were calculated. The relative potency for the control was calculated using the following equation: Relative Potency=(RefStd EC50GM)/(Control EC50)×100%.

The samples were calculated in the same manner. Results of the assay were determined by the following equation: Relative Potency=(RefStd EC50GM)/(Test sample EC50)×100%.

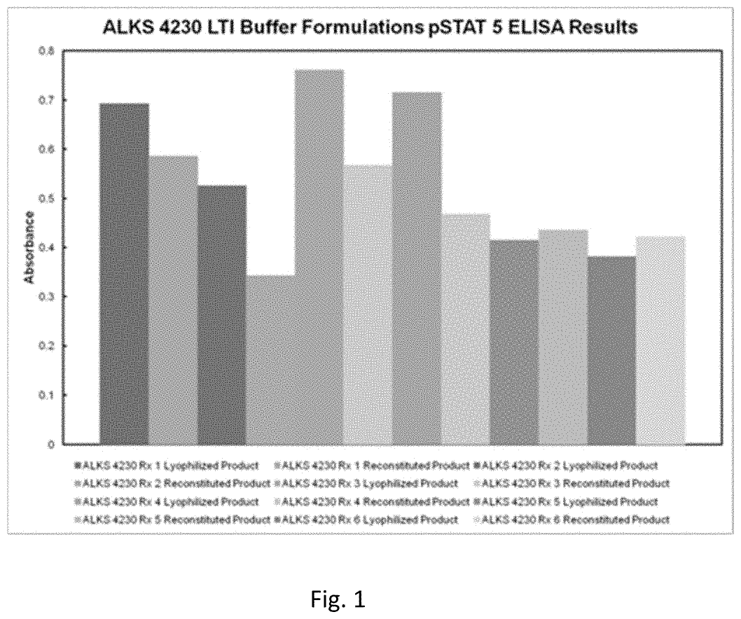

The results of the HH cell line-based activity assay demonstrated that Polypeptide A maintained activity in the various formulations tested (FIG. 1).

### Stability Tests Results

The results of the foregoing stability characterization revealed that formulation 4 was superior to the other formulations tested at maintaining pH and protein stability of Polypeptide A.

### Example 2—Screen of Different Buffer Components and their Effect on Stability

Several buffers appropriate for pharmaceutical use in the pH range 6.0-7.0 were screened for stabilizing effect on Polypeptide A using Differential scanning calorimetry and an OD350 time course assay for screening based on thermal stress, and micro-flow imaging for screening based on shake stress.

Polypeptide A (initially at 11.6 mg/mL in PBS buffer) was dialyzed into one of five buffers at pH 6.0, 10 mM histidine, 10 mM citrate, 100 mM citrate, 10 mM phosphate, or 10 mM succinate. Dialysis was performed in Slide-A-Lyzer cassettes with a 10,000 Da molecular weight cutoff and a 3 mL capacity, at 4° C., and at a ratio of 1:500+ dialysate volume to buffer volume, with 2 buffer changes at 4+ hour intervals, and incubation overnight before dialysate recovery. The dialysate was diluted to 1.0 mg/mL Polypeptide A with filtered (0.2 μm) dialysis buffer. Assays were then performed on these samples, in triplicate. For Micro-flow Imaging, 3 mL of each sample in duplicate were added to 5 mL vials, and the vials were shaken for 3 days at 300 RPM, then duplicates were pooled. Three mL of buffer was also shaken under the same conditions for buffer subtraction and blanking.

### Differential Scanning Calorimetry (DSC)

DSC experiments were performed in triplicate on all samples at an Polypeptide A concentration of 1 mg/mL, using a MicroCal capillary differential scanning calorimeter equipped with an autosampler. Samples were heated from 10 to 90° C. at a rate of 60° C./hour and thermograms were recorded. The sample and reference cells were washed between each injection. Data were analyzed using the MicroCal analysis package add-on in Origin 7, and interpolated to the same x-axis values so that averaging could be performed.

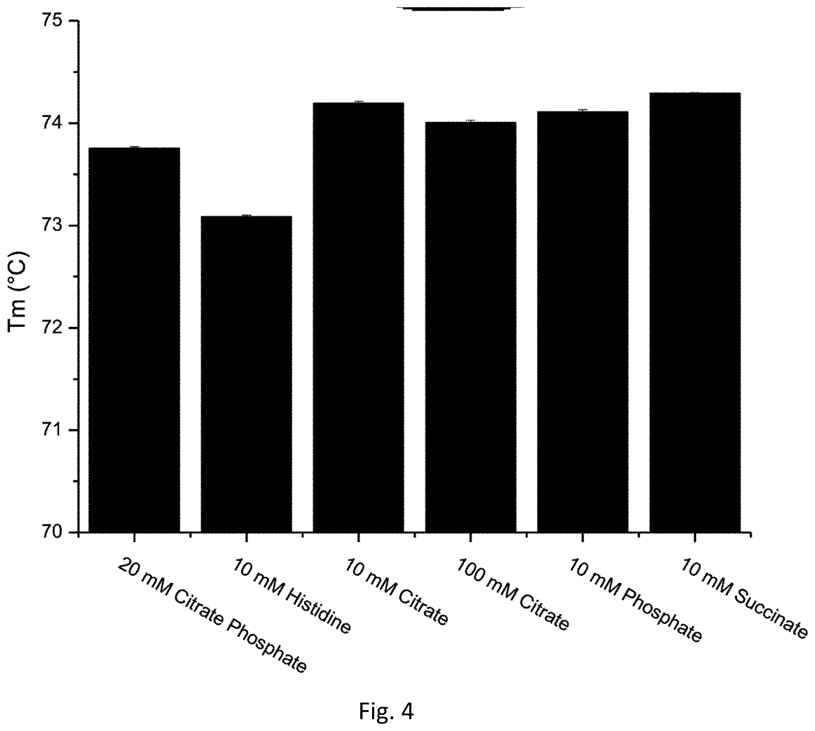

FIG. 4 shows the Tₘ values of the thermograms as a function of buffer composition. The Tₘ value of 10 mM citrate was the highest of all samples and the 10 mM histidine buffer had a relatively lower Tₘ value.

### OD350 Time Course (Kinetic) Assay

OD350 time course (kinetic) assays were performed in triplicate on a Cary 100 UV-Visible spectrophotometer using a volume of 260 μL of protein solution per cuvette. The optical density at 350 nm was measured every five minutes with an averaging time of two seconds, while the temperature was held at a constant value of 72° C. All data were imported into Origin lab and interpolated to the same x-axis values so that averaging could be performed

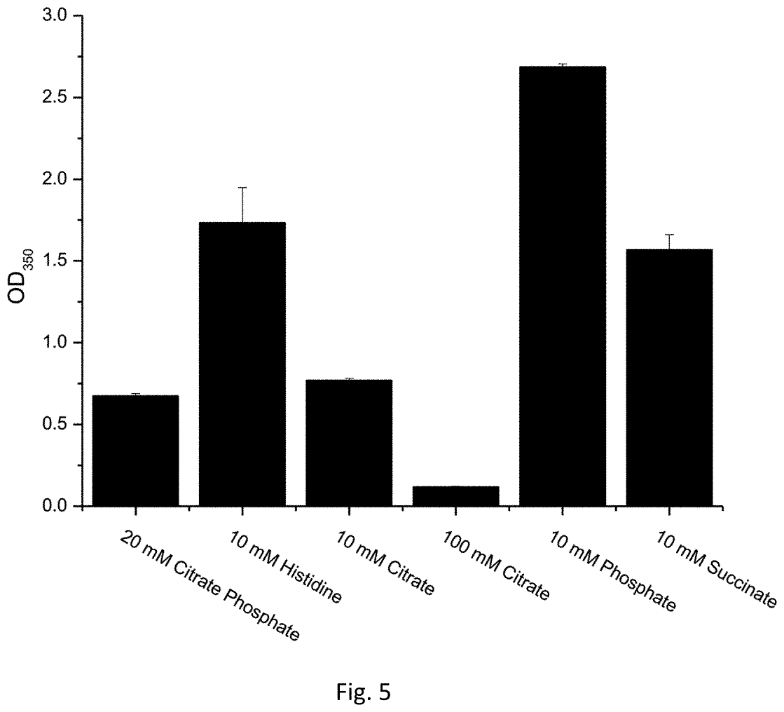

OD350 values were collected in triplicate for Polypeptide A samples every five minutes for eight hours at a constant temperature of 72° C. FIG. 5 shows average OD350 values after 8 hours. After 8 hours at 72° C., samples containing citrate buffers had the lowest OD350 value.

### Micro-Flow Imaging

Subvisible particles in the range of 2 to 70 μm were examined using a MFI DPA-4200 (Protein Simple, Santa Clara, Calif.) system with a 100 μm silane coated flow cell. The instrument was calibrated using 10 μm polystyrene particle standards (Duke) prior to measurements. Measurements were made in triplicate at ambient temperature for all samples. The cell was flushed with particle free water and Illumination was optimized using particle free water prior to all measurements. The samples were carefully drawn up in a low protein binding, filter-tip pipette (Neptune Scientific) and analyzed using a flow rate of 0.2 mL/min. The purge volume for each measurement was 0.4 mL, and 0.6 mL of sample was analyzed.

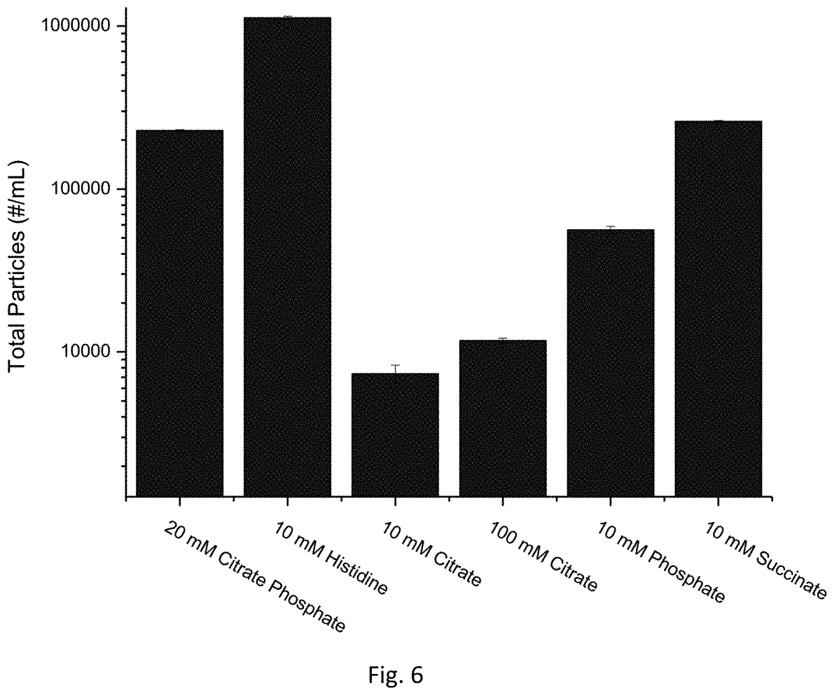

MFI was performed on all Polypeptide A samples in triplicate after 3 days of shaking at 300 RPM, and the results are shown in FIG. 6. Samples in 10 mM histidine had the highest total subvisible particle concentration after 3 days of shaking, and samples in 10 mM citrate had the lowest total subvisible particle concentration.

### Conclusions for Buffer Screening

Samples of Polypeptide A in 10 mM histidine had the lowest thermal stability, highest initial OD350 with time at 72° C., and the highest total subvisible particle concentration after 3 days of shake-stress. Samples in citrate buffer had the lowest OD350 with time at 72° C., the lowest total particle concentration after 3 days of shake stress, and among the highest thermal stability of samples in DSC data.

### Example 3—Comparison of Histidine Buffer Against Citrate with Sucrose and Citrate with Sucrose and Polysorbate 20

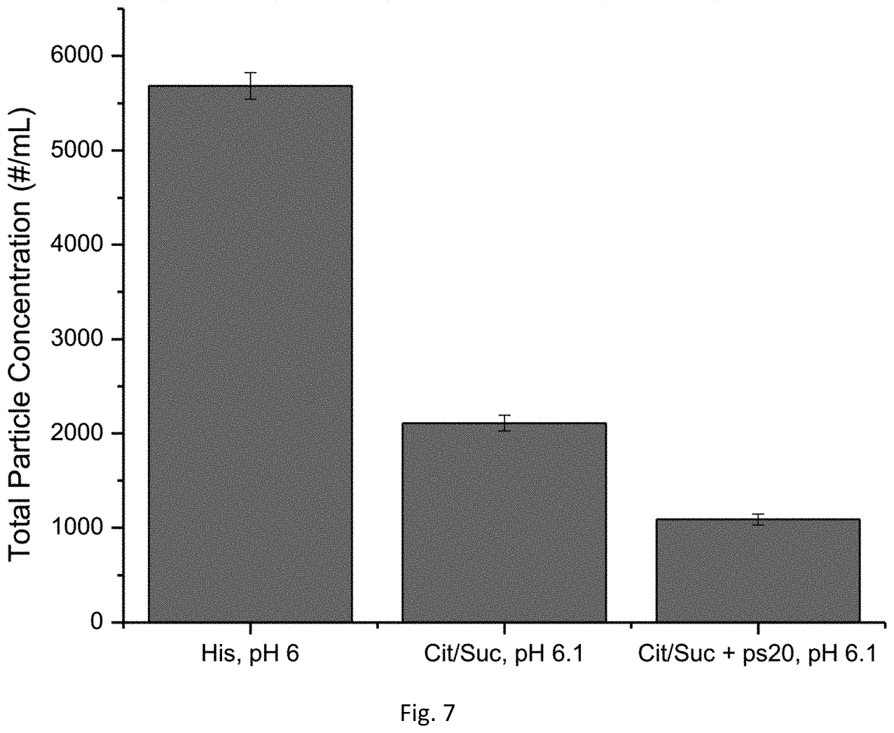

In an effort to determine the effect of the presence of polysorbate 20 in 3 mg/mL citrate, 50 mg/mL sucrose, pH 6.1 on the total subvisible particle concentration as a function of shake stress, micro-flow imaging was performed. The buffers tested were 10 mM histidine, 3 mg/mL citrate with 50 mg/mL sucrose, and 3 mg/mL citrate with 50 mg/mL sucrose and 0.01% polysorbate 20. Total subvisible particle concentration was measured in triplicate for all Polypeptide A samples by microflow imaging after 3 days of shaking at 300 RPM; and the results are shown in FIG. 7. The total subvisible particle concentration was lowest in samples with polysorbate 20.

1. A composition comprising:
a) about 1 mg to about 50 mg of a polypeptide comprising a circularly permuted IL-2 fused to the extracellular portion of an IL-2Rα chain;
b) sucrose;
c) citrate buffer; and
d) polysorbate 20.

2. The composition of claim 1, wherein the polypeptide comprises an amino acid sequence having at least 95% identity to SEQ ID NO: 1.

3. (canceled)

4. The composition of claim 1, wherein the composition comprises:
about 2.2 mg of the polypeptide, about 11 mg of the polypeptide, or about 33 mg of the polypeptide;
about 100 mg to about 120 mg sucrose, or about 110 mg sucrose;
about 4.0 mg to about 6.0 mg of citrate anion, or about 5.0 mg citrate anion; and/or
about 0.20 mg to about 0.24 mg polysorbate 20, or about 0.22 mg polysorbate 20.

5-10. (canceled)

11. The composition of claim 1, wherein the composition comprises citric acid and sodium citrate tribasic dihydrate in a mass ratio of citric acid:sodium citrate tribasic dihydrate of between about 1:10 to about 1:2, optionally comprising citric acid and sodium citrate tribasic dihydrate in a mass ratio of citric acid:sodium citrate tribasic dihydrate of about 1:9 or about 1:2.

12-15. (canceled)

16. The composition of claim 1, wherein the composition is a lyophilized cake, optionally wherein:
dissolution of the lyophilized cake in water results in an aqueous solution with a pH of about 5.5 to about 6.5, or about a pH of about 6.1;
dissolution of the lyophilized cake in water results in an aqueous solution with an osmolality of about 160 to about 230 mOsm/kg, or about 179 mOsm/kg; and/or
dissolution of the lyophilized cake in a sodium chloride solution results in an aqueous solution with an osmolality of about 240 to about 340 mOsm/kg, optionally wherein the sodium chloride solution comprises about 0.1% NaCl to about 0.5% NaCl, about 0.12% NaCl to about 0.41% NaCl, or about 0.02 M NaCl to about 0.07 M NaCl.

17-24. (canceled)

25. The composition of claim 1, wherein the composition is an aqueous solution, optionally wherein:
the composition comprises about 1 mg/mL of the polypeptide, optionally wherein the composition is a 2.2 ml aqueous solution comprising about 2.2 mg of the polypeptide;
the composition comprises about 5 mg/mL of the polypeptide, optionally wherein the composition is a 2.2 ml aqueous solution comprising about 11 mg of the polypeptide; and/or
the composition comprises about 15 mg/mL of the polypeptide, optionally wherein the composition is a 2.2 ml aqueous solution comprising about 33 mg of the polypeptide.

26-31. (canceled)

32. The composition of claim 25, wherein:
the composition comprises about 45 mg/mL to about 55 mg/mL sucrose, or about 50 mg/mL sucrose;
the composition comprises about 10 mM to about 20 mM citrate buffer, or about 12 mM citrate buffer;
the citrate buffer is formed by the combination of 2.03 mg/mL sodium citrate tribasic dihydrate and 0.97 mg/mL citric acid monohydrate in the aqueous solution
the composition comprises about 0.09 mg/mL to about 0.11 mg/mL polysorbate 20, or about 0.1 mg/mL polysorbate 20;
the pH of the composition is about 5.5 to about 6.5, or about 6.1;
the osmolality of the composition is about 160 to about 230 mOsm/kg, or about 179 mOsm/kg; and/or
the aqueous solution comprises about 0.03 mg/mL of the polypeptide to about 0.2 mg/mL of the polypeptide.

33-43. (canceled)

44. A lyophilized composition made by lyophilizing the composition of claim 25.

45. The composition of claim 1, wherein the composition is a single unit dose of the polypeptide.

46. An article of manufacture comprising the composition of claim 1, optionally which is a glass vial.

47. (canceled)

48. A method of making a lyophilized composition, the method comprising lyophilizing the aqueous solution of claim 25.

49. A method of making an aqueous composition, the method comprising dissolving the composition of claim 16 in an aqueous solvent optionally wherein the aqueous solvent comprises water for injection or a sodium chloride solution, optionally wherein the sodium chloride solution comprises about 0.1% NaCl to about 0.5% NaCl, about 0.12% NaCl to about 0.41% NaCl, about 0.02 M NaCl to about 0.07 M NaCl.

50-55. (canceled)

56. The method of claim 49, wherein the pH of the aqueous composition is adjusted to about 6.1, optionally wherein the pH of the aqueous composition is adjusted to about 6.1 with a base, optionally wherein the base is sodium hydroxide.

57. (canceled)

58. (canceled)

59. The method of claim 49, wherein the aqueous composition is further diluted with an aqueous solution comprising about 1% (w/w) of a surfactant, optionally wherein:
the surfactant is polysorbate 20; and/or
the aqueous solution further comprises about 0.1% (w/w) citric acid monohydrate, 0.2% (w/w) sodium citrate tribasic dihydrate, and 98.7% (w/w) water for injection.

60. (canceled)

61. (canceled)

62. A method of activating natural killer cells (NK) cells in a subject, the method comprising administering to the subject an effective amount of the composition of claim 25.

63. A method of treating cancer in a subject in need thereof, the method comprising administering to the subject an effective amount of the composition of claim 25, optionally wherein the cancer is renal cell carcinoma, melanoma, ovarian cancer, or lung cancer, and/or the cancer comprises a refractory solid tumor.

64. (canceled)

65. (canceled)
